(19) United States
(12) Patent Application Publication
(10) Pub. No.: US 2008/0312723 A1
(43) Pub. Date: Dec. 18, 2008

(54) Apparatus and Method for Inducing Suspended Animation Using Rapid, Whole Body, Profound Hypothermia

(75) Inventors: Ralph E. Gill, Ligonier, PA (US); William Sylvester Novak, Monroeville, PA (US); Michael Nicholas Pitsakis, Coral, PA (US); Vladimir Zhuze, Indiana, PA (US)

Correspondence Address: THE WEBB LAW FIRM, P.C., 700 KOPPERS BUILDING, 436 SEVENTH AVENUE, PITTSBURGH, PA, 15219, US

(73) Assignee: ARDIEM MEDICAL, INC., Indiana, PA (US)

(21) Appl. No.: 11/597,157

(22) PCT Filed: May 26, 2005
(86) PCT No.: PCT/US05/18705

§ 371 Date: Jul. 17, 2008

Related U.S. Application Data
(60) Provisional application No. 60/574,689, filed on May 26, 2004.

(51) Int. Cl. A61F 7/00

(52) U.S. Cl. 607/105

(57) ABSTRACT

Disclosed is a transportable apparatus that maintains a large volume of a cold solution at a preset low temperature and rapidly delivers it to an irresuscitable patient's thoracic aorta at a preset and controlled flow rate, while monitoring pressure and eliminating the introduction of air embolisms into the patient, for the purpose of inducing profound hypothermia and suspended animation until proper treatment of the patient is available. The induction of suspended animation is intended for preservation of viability of the brain, heart, and organs for subsequent repair and resuscitation or for organ harvesting. The procedure may be performed by emergency personnel on the scene of an accident and/or while the patient is being transported in an emergency vehicle, or while in a hospital.

### BACKGROUND OF THE INVENTION

1. Field of the Invention

The invention relates to the use of emergency therapeutic hypothermia. In particular, the present invention relates to providing transportable, emergency, cooling capabilities to rapidly induce profound hypothermia and suspended animation at the point-of-injury for irresuscitable patients.

2. Description of Related Art

Standard cardiopulmonary-cerebral resuscitation fails to achieve spontaneous circulation in a large percentage of normovolemic sudden cardiac arrest cases outside of hospitals and in nearly all cases of penetrating truncal trauma who exsanguinate rapidly due to cardiac arrest. Few such resuscitable patients reach the hospital in time to be saved. Speed in these and other trauma cases is crucial. The critical maximal times for complete ischemia from cardiac arrest that can be tolerated at normothermia are approximately five minutes for the brain, twenty minutes for the heart, and thirty minutes for the viscera. Among cardiopulmonary-cerebral resuscitation innovations since the 1960s, automatic external defibrillation, hypothermia, portable emergency cardiopulmonary bypass, and suspended animation have potential for clinical breakthrough effects. Current studies indicate that a rapid induction of profound hypothermia to induce suspended animation in a patient without respiration or circulation preserves the viability of the heart, brain, and other internal organs. Profound hypothermia for suspended animation consists of the rapid reduction of a patient's core body temperature to below 10° C., with or without drugs, for preservation of viability of the brain, heart and organs for later organ repair or organ harvesting. With a suspended animation method, the organs of the victim are preserved by the rapid cold flush, and the procedure gives the medics more time for transport and hemostasis, which can then be followed by delayed resuscitation once the victim arrives at the hospital. Suspended animation, induced at the point of injury, could ultimately benefit any irresuscitable patient who, given sufficient time for transportation and restorative surgery, could be resuscitated to a normal physiological condition. If the patient cannot be resuscitated, suspended animation increases the time for organ harvesting.

Prior attempts to preserve bodily organs during ischemia and/or anoxia for later resuscitation or harvesting have been limited. An example is provided by U.S. Pat. No. 5,709,654, which shows an apparatus for treating ischemic and anoxic brain injuries associated with cardiac arrest. The described apparatus circulates chilled oxygenated fluid to the brain by catheterizing the external carotid arteries, while continuing resuscitation efforts to restore circulation and respiration. The apparatus can be used to maintain viability of other bodily organs, but is primarily focused on maintaining viability of the brain during resuscitation. The apparatus chills about three liters of a resuscitation solution to a temperature of 4° C. to 5° C. The solution is then oxygenated and circulated through catheters to the brain during resuscitation efforts. Use of the apparatus is stopped when the patient's heart is restarted and circulation is restored. If the patient is not revived or is declared not resuscitable, the apparatus can be used to target another organ for harvesting.

This approach is not suitable to induce whole body suspended animation for the purpose of providing time for transportation and delayed resuscitation. The solution volume is insufficient to rapidly drop the core body temperature enough for suspended animation and can only target the brain or a single organ for harvesting for about 30 minutes.

### SUMMARY OF THE INVENTION

Suspended animation is accomplished using a rapid, one way, aortic flush of a cold solution, flushing and replacing blood, to specific organs, such as the brain, or through the entire body to induce profound hypothermia in irresuscitable patients. The present invention maintains 40 to 60 liters of pre-chilled solution at a temperature of −5° C. to 10° C. The apparatus can lower an adult man's core temperature to 10° C. or below within a few minutes. The compact size of the apparatus allows transportation in an emergency vehicle so the procedure can then be performed at the point-of-injury, increasing the patient's chance of survival. The present invention chills the entire body with a flush, not circulation, lowers the core body temperature, not just a targeted organ, to below 10° C., and induces a state of suspended animation for two or more hours.

While prior art devices may be appropriate for the particular purpose cited, or for general use, they would not be as suitable for the purposes of the present invention as disclosed herein. Accordingly, the present invention is an apparatus that can rapidly induce profound hypothermia for a state of suspended animation, a core body temperature below 10° C., in approximately ten minutes.

Described in its preferred embodiment, the invention provides a transportable, emergency, cooling apparatus, along with cooling methodologies and clinical applications, to rapidly induce whole body or regional, profound hypothermia by means of a one way aortic flush, to lower the core body temperature of an irresuscitable patient to below 10° C. The apparatus is comprised of a means for maintaining a large volume of a cold flushing solution at a predetermined temperature (−5° C. to 5° C.), a means for delivering the cold solution via a catheter, a disposable infusion tubing set, and pump, and a means for controlling the flow rate and temperature of the solution. The apparatus can be configured for use in a hospital or in the field at the point-of-injury through transport in an ambulance, rescue vehicle, or other transport vehicle. Additionally, the apparatus includes a means for changing the solution containers within the cold chamber of the apparatus without interrupting the flow of solution to the victim, thus allowing for the delivery of an unlimited volume of cold solution. Included in the apparatus is a means for monitoring and displaying the core body temperature of the patient. The apparatus is used to induce suspended animation in irresuscitable patients resulting from a variety of emergency medical situations, in particular cardiac arrest.

Profound hypothermia and suspended animation is induced in a patient through several access techniques, depending on the specific situation. The cold solution line must be connected to one of three catheter access strategies for catheter placement within the aorta. These three aorta access strategies include placements via thoracotomy, peripheral, or transthoracic approaches to the aorta depending on the patient condition and assessment of approach options. The cold flush aortic catheter placed in each of the approaches must be connected to the cold solution line via an appropriate connector, depending on the catheter's type and size. The vascular access approaches for inducing suspended animation requires rapid vascular access to the thoracic aorta via several specific approaches for placement of the cold flush catheter. These approaches to the thoracic aorta include:

(a) Direct access to the thoracic/descending aorta for the cold flush catheter via a left thoracotomy performed by the surgeon that permits visualization of the descending aorta in the chest, location of a catheter insertion point on the aorta and placement of the cold flush catheter directly into the thoracic/descending aorta followed by inflation of a sealing balloon. A specific thoracotomy access catheter must be available for this thoracotomy approach that is able to seal the aortotomy site with targeted delivery of the cold solution, first to the brain and heart, then globally.

(b) Femoral artery access to the thoracic/descending aorta for the cold flush catheter via right or left femoral artery location and insertion of the catheter into the either femoral artery with catheter advancement and placement in the thoracic/descending aorta. A femoral artery catheter fitted with occluding balloon must be used that permits targeted cold solution delivery, first to the brain and heart, and then globally when the occluding balloon on the catheter is deflated.

(c) Carotid artery access to the aortic arch and thoracic aorta for the cold flush catheter via the right or left common carotid artery location and insertion of the catheter into the carotid artery with catheter advancement and placement into the thoracic/descending aorta. The carotid artery catheter must be fitted with an occluding balloon to permit targeted cold solution delivery, first to the heart and brain, then globally with deflation of the occluding balloon.

(d) Subclavian artery access to the aortic arch and thoracic aorta for the cold flush catheter via the right or left subclavian artery location and insertion of the catheter into the subclavian artery below the clavicular bone with catheter advancement and placement into the thoracic/descending aorta. The subclavian artery catheter must be fitted with an occluding balloon to permit targeted cold solution delivery, first to the heart and brain, then globally with deflation of the occluding balloon.

(e) Brachial/auxiliary artery access in the arm to the aortic arch and thoracic aorta for the cold flush catheter via the right or left brachial/auxiliary artery location and insertion of the catheter into the brachial/auxiliary artery with catheter advancement and placement into thoracic/descending aorta. The brachial/auxiliary artery catheter must be fitted with an occluding balloon to permit targeted cold solution delivery, first to the heart and brain, then globally with deflation of the occluding balloon.

(f) Direct access to the ascending aorta and aortic arch for the cold flush catheter via a transthoracic approach that permits the guided insertion of a cold flush catheter through the right chest wall, parasternally, directly into the ascending aorta with catheter advancement through the aortic arch and placement in the thoracic/descending aorta. A specific transthoracic access catheter is needed to seal the aortotomy site and provide an occluding balloon to permit targeted cold solution delivery, first to the heart and brain, then globally with deflation of the occluding balloon.

(g) Other peripheral artery access and approaches that easily and adequately permit insertion of the cold flush catheter and placement in the thoracic/descending aorta. Periphery artery catheters fitted with occluding balloons must be used that permit targeted cold solution delivery, first to the heart and brain, then globally with deflation of the occluding balloon.

After achieving catheter placement, connection to the cold solution line, and installment of the disposable set, the apparatus pumps the cooling solution to the patient at a selectable flow rate. The cooling solution is first delivered to the heart and brain, and then to the remainder of the body. As the solution is delivered to the patient's circulatory system, blood is flushed from the system and replaced by the cooling solution. The apparatus continues to flush cooling solution through the patient's circulatory system until the core body temperature decreases to the desired level. If more solution is needed, new pre-cooled solution bags or canisters replace the empty solution bags or canisters in the apparatus cooling chamber.

The flush solution may include cold saline, with or without additives, that enhance protection during the induction phase of cooling as well as the recovery or rewarming phases. These additives include oxygen saturated in the solution, oxygen carrying blood substitutes or hemoglobin-based blood substitutes, free radical scavengers like tempol, glucose, and similar acting compounds, or energy substrate sources and compounds that mitigate reperfusion injury, as well as compounds that induce metabolic down-regulation or hibernation like states, like hydrogen sulfide, enhancing the effects of cooling.

It is therefore an object of this invention to induce profound hypothermia and suspended animation in a very rapid manner by means of a one way aortic flush using a required volume of cooling solution, with the capability of an unlimited volume of solution, to lower an adult man's core temperature to 10° C. or below in approximately ten minutes.

It is another object of the invention that it is transportable and capable of functioning in an emergency vehicle for immediate use at the point-of-injury following an irresuscitable event. Emergency personnel can immediately initiate the induction of profound hypothermia at the scene prior to transportation to a hospital, or in route to the hospital.

It is a further object of the invention that the apparatus can be used in both an emergency vehicle and in a hospital setting. Hospital and emergency personnel can use the apparatus independent of the emergency vehicle when required.

It is yet another object of the invention that up to sixty (60) liters of cooling solution, contained in bags or canisters, may be maintained at its pre-chilled temperature of −5° C. to 5° C. while the apparatus is transported in the emergency vehicle to the point-of-injury.

It is another object of the invention that while the cooling solution is delivered to the patient, the apparatus controls flow rate and pressure, and eliminates the introduction of air embolisms into the patient.

It is still another object of the invention to function with predetermined safety limits, alarming the user of conditions during a procedure such as low cooling solution, alerting the user to install new cooling solution.

Further objectives of the invention will become apparent from consideration of the drawings and the ensuing detailed description of the invention which follows. A person skilled in the art will realize that other embodiments of the invention are possible and that the details of the invention can be modified in a number of respects, all without departing from the inventive concept. Thus, the following drawings and detailed description are to be regarded as illustrative in nature and not restrictive.

### BRIEF DESCRIPTION OF THE DRAWINGS

The features of the invention will be better understood by reference to the accompanying drawings, which illustrate presently preferred embodiments of the invention.

FIG. 1 is a system diagram of the apparatus described herein and illustrates the preferred embodiment;

FIG. 2 is a layout of the disposable infusion tubing set; and

FIG. 3 is a system control block diagram.

### DETAILED DESCRIPTION OF THE PREFERRED EMBODIMENTS

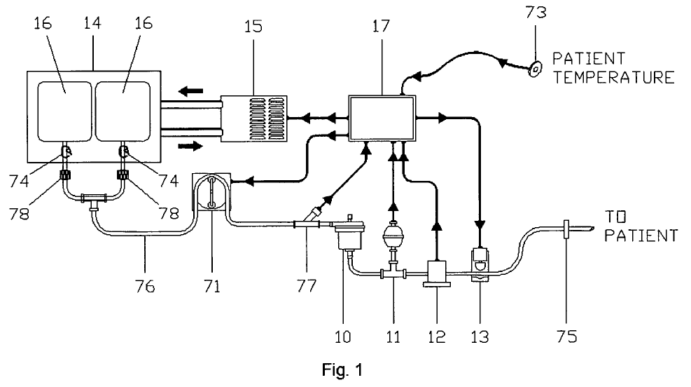

Referring to FIGS. 1-3, a preferred embodiment of the apparatus is disclosed. The apparatus consists of the profound hypothermia apparatus and a disposable sterile infusion tubing set. Operation of the invention involves cannulation of the patient in a manner that will introduce the solution into the patient in the desired location and direction, connecting the sterile infusion tubing set to the apparatus and loading the sterile aortic flushing solution into the apparatus. The solution may be delivered to the patient through several access techniques, depending upon the specific situation. The variations do not materially affect the apparatus design or configuration. The delivery tubing set is primed, bubbles are evacuated, and the tubing is connected to the cannula. The patient temperature sensor is installed, the solution flow rate is selected from the control panel, and the delivery pump is started. The delivery of the cold flush solution is continued until the desired core body temperature of the patient is attained.

FIG. 1 shows the system diagram and main components of the apparatus. Flush solution bags (or canisters) 16 are installed in a cold chamber 14 which is chilled by an evaporator and a refrigeration unit 15. The cold flush solution exits the solution bags 16 through a sterile tubing 76 and fitting 78 to an infusion pump 71. The infusion pump assembly 71 pumps the cooling solution through a bubble trap/filter 10 to remove air bubbles, and a pressure isolator 11 is used to isolate the blood from the pressure sensor. The pumped cooling solution exits the pressure isolator 11 into a bubble detector 12 and a tubing occluder 13, and to the patient through a connector 75, connecting a previously installed cold flush catheter to the sterile tubing 76. If the bubble detector 12 detects bubbles in the flow of blood, the tubing occluder 13 is activated by the system controls to stop the flow of blood through the apparatus. An electronic control unit 17 monitors the patient's core body temperature from a patient temperature sensor 73, cooling solution temperature and pressure, flow rate, and other parameters for system control and safety, and controls the data output to the user interface and system inputs from the user.

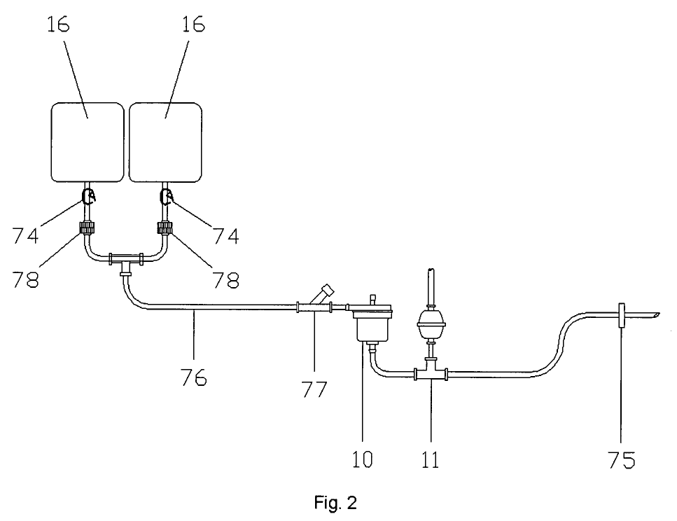

FIG. 2 shows the disposable sterile infusion tubing set. The disposable tubing is set sterile and heparin bondable and consists of the solution bag(s) or canister(s) 16 containing the cold flush solution; pinch clamps 74 for occluding the tubing while replacing solution bags; connection fittings 78 for connecting the solution bags 16 to the sterile tubing 76; an injection port 77 for inserting a myocardial temperature probe used in monitoring the inflow solution temperature; a bubble trap/filter 10 capable of removing air bubbles larger than 20 μL; a pressure isolator 11 used to isolate the cooling solution from a pressure sensor; and a connector fitting 75 for connecting the sterile tubing 76 to the cold flush catheter.

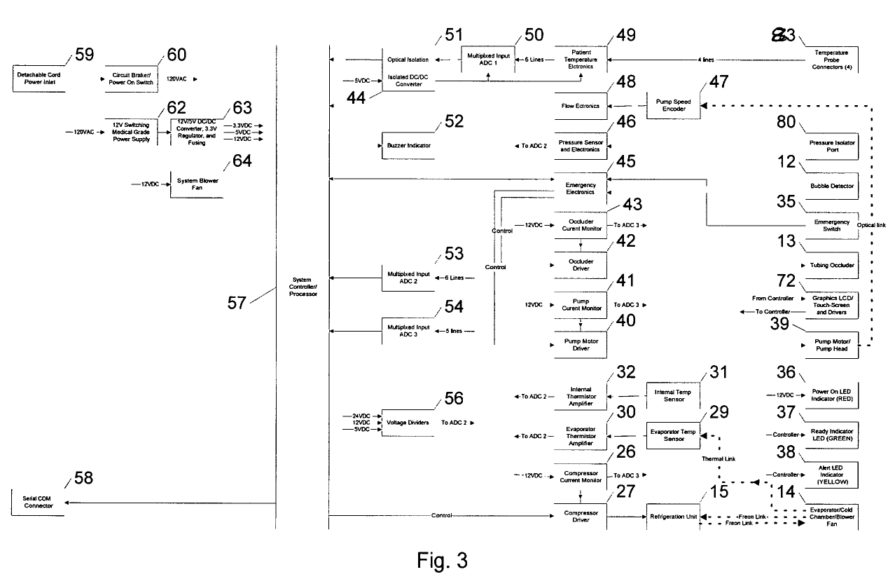

FIG. 3 shows the system control block diagram. The system includes a power inlet 59 where a detachable hospital grade power cord connects a 2-pole circuit breaker 60 that also serves as a power switch, and a standard serial COM connector 58 for providing communication means and data upload to a computer, both accessible on the rear panel. A fan 64 is also mounted on the rear panel to provide forced cooling air and prevent the temperature inside the unit from rising above 40° C. A 12 VDC medical grade switching power supply 62 provides 12 VDC to a 12V/5V DC/DC converter and regulator 63, which supplies 5 VDC and 3.3 VDC to the electronics, to the pump motor, and to the occluder solenoid. The apparatus can also entirely operate on 12 VDC for application in a battery operated portable unit.

The front panel contains four temperature probe connectors 83 for connecting up to four temperature probes for sensing solution bag temperature, patient temperatures, inflow temperature, and/or outflow temperature; a pressure port 80 for connecting the pressure isolator 11 to a pressure sensor 46; an ultrasonic bubble detector 12; a push-button emergency switch 35 for emergency shut down; physical access to the head of a pump 39; physical access to the cold chamber 14; user interface graphics and drivers 72 for user interface; a LED power-on indicator 36; a LED ready indicator 37 that indicates the apparatus is ready for a procedure; and a LED alert indicator 38 that is illuminated whenever a fault or alert is detected and a message is displayed.

The compressor of the refrigeration unit 15 is turned on/off by a compressor driver 27 under command of a system controller/processor 57. A standard thermistor 29, is placed in the cold chamber 14 and, after amplification by an evaporator thermistor amplifier 30, is routed to an analog to digital converter, multiplexed input ADC 2 53 for the purpose of monitoring the cold chamber temperature. Similarly, another standard thermistor, an internal ambient temperature sensor 31, is interfaced to an internal ambient thermistor amplifier 32 and also routed to multiplexed ADC 2 53 for monitoring the temperature inside the apparatus. The pump motor/head 39 is driven by the pump motor drive under control of the system controller/processor 57 in a pulse-width modulation fashion that sets the speed of the motor and the flow rate from 0-100% if an emergency electronics circuit 45 has not been activated. Flow measurement is accomplished by an encoder wheel and an opto-isolator, a pump speed encoder 47, attached to the back shaft of the motor. The combination generates pulses of frequency proportional to pump speed and, by extension, to the solution flow rate. The output of the encoder 47 is fed to the flow electronics 48 for conditioning and shaping, and then to the system controller/processor 57 for measurement. The contraction of the tubing wall due to the cold solution has a negligible effect on accuracy. This effect, plus the control algorithm deviation, amount to less than ±10% deviation in flow rate. The tubing occluder 13 is driven by an occluder driver 42 via the emergency electronics 45 and is commanded primarily by the bubble detector 12 or the emergency switch 35 and secondarily by the system controller/processor 57 for testing only. The emergency electronics 45 reacts directly to signals from the bubble detector 12 or the emergency switch 35 while flagging the system controller/processor 57. The system controller/processor 57 has means to disable the bubble detector during priming of the tubing to enable the apparatus for a procedure, and also has means to reset the emergency electronics after an emergency stop condition is cleared. The pressure sensor and electronics 46 provide an analog signal proportional to pressure that is fed to the multiplexed ADC 2 53. The patient temperature electronics 49 contains four thermistor amplifiers. The four temperature signals, plus two internal voltages for diagnostics, are fed to ADC 1 50 for monitoring. Electrical isolation is required for this section and is accomplished by the optical isolators in optical isolation 51 and by a transformer isolated DC/DC converter depicted by the DC/DC converter/regulator 44. The DC currents of the occluder solenoid, pump motor, and bypass valve stepper motor are monitored for diagnostics via the occluder current monitor 43 and the pump current monitor 41. The current-related voltages of these blocks are fed to multiplexed input ADC 3 54 for monitoring and fault detection. These elements also provide individual circuit fusing. In addition, the scaled down versions, by a voltage divider 56, of the supply voltages 12 VDC and 5 VDC are fed to multiplexed input ADC 3 54 for monitoring and fault detection.

What is claimed is:

1. A profound hyperthermia apparatus which provides emergency cooling to rapidly induce whole body or regional profound hypothermia by means of a one way aortic flush of a large volume of cold solution to lower the core body temperature of an irresuscitable patient to below 10° C. for a state of suspended animation, said apparatus comprising:
a cold chamber for storing and maintaining the temperature (−5° C. to 10° C.) of pre-chilled solution bags or canisters, such that the solution bags or canisters can be easily exchanged when emptied, allowing delivery of an unlimited volume of solution;
a pump and motor for controlling the flow rate and pressure at which the solution is delivered;
a pressure transducer and a flow rate transducer which monitor the flow of solution and provide data to system electronics for controlling the pump;
a bubble trap/filter which deters small bubbles from entering the patient's circulatory system;
a bubble detector that monitors the solution for clusters of small bubbles or a large bubble that was not removed by the bubble trap/filter;
a sterile disposable infusion tubing set for delivering the cold flush solution; and
an electronics assembly and software for monitoring sensors, managing inputs, controlling pump operations, and displaying data, status, and alarms to an operator.

2. The profound hypothermia apparatus as recited in claim 1, wherein the apparatus is easily transported between locations by an average adult and is transportable by emergency vehicle.

3. The profound hypothermia apparatus as recited in claim 2, wherein the apparatus can be located, powered, and operated in an emergency vehicle or in a hospital or clinic.

4. The profound hypothermia apparatus as recited in claim 1, wherein the apparatus can lower a patient's core body temperature to the range of 0° C. to 10° C.

5. The profound hypothermia apparatus as recited in claim 4, wherein the apparatus can rapidly induce profound hypothermia and suspended animation by lowering an irresuscitable patient's core body temperature to 10° C. in ten minutes or less.
